(19) United States
(12) Patent Application Publication
(10) Pub. No.: US 2015/0196913 A1
(43) Pub. Date: Jul. 16, 2015

(54) MICROFLUIDIC CHIP FOR CAPTURING CELLS AND PREPARING METHOD THEREOF

(71) Applicant: WUHAN YZY BIOPHARMA CO., LTD., Wuhan, Hubei (CN)

(72) Inventors: Kan Liu, Wuhan (CN); Shengxiang Wang, Wuhan (CN); Nangang Zhang, Wuhan (CN); Pengfei Zhou, Wuhan (CN)

(21) Appl. No.: 14/418,122

(22) PCT Filed: Nov. 14, 2012
(86) PCT No.: PCT/CN2012/084603

§ 371 Date: Jan. 29, 2015

(30) Foreign Application Priority Data
Nov. 8, 2012 (CN) 201210442637.7

(51) Int. Cl. B01L 3/00; G01N 33/50

(52) U.S. Cl. CPC B01L 3/502707; G01N 33/5044; B01L 2300/16; B01L 2300/12

(57) ABSTRACT

A microfluidic chip (100) for capturing cells (200) and preparing method thereof. The chip (100) comprises an upper rigid material (10) and a lower rigid material (20), a channel (30) provided between the upper rigid material (10) and the lower rigid material (20). The channel has an inlet (32) and an outlet (34). At least one of the upper rigid material (10) and the lower rigid material (20) is made of transparent material. The channel (30) has a height decreasing gradually from the inlet (32) to the outlet (34), thus a wedge shape is formed, or a portion of the channel has the wedge shape. The lowest portion of the channel (30) has a dimension approximate to or less than that of at least one of target cells. The chip (100) could quickly, effectively separate and enrich cells with various sizes and specific molecular expressions.

### TECHNICAL FIELD

The present application relates to an instrument for capturing cells and the preparation method thereof, in particular, it relates to a microfluidic cell capture chip for the separation, enrichment and recognition of circulating tumor cells and the preparation method thereof, this microfluidic cell capture chip can be used as an advantageous instrument for diagnosis of tumor, adjuvant therapy and biochemical analysis and research.

### BACKGROUND

Nowadays, diagnosis of tumor is usually carried out based on a number of pathological conditions. Such methods, e.g., biopsy or digital rectal examination for rectal cancer are often carried out by invasive analysis, and they can cause a certain degree of trauma, so these methods are unfavorable for patients.

On the other hand, the examination can be carried out by testing bio-molecular markers in peripheral blood, such as determining serological parameters (PSA dose), because this kind of examination is not satisfactory on sensitivity and specificity, it cannot make good effect on cancer diagnosis and treatment.

For many cancer patients, the death is mainly caused by metastasis tumor. After the primary tumors of a patient is surgically resected, it is difficult to reflect the following treatment status, identify the metastasis tumor, and make guidance for subsequent chemotherapy timely by means of current detection methods, therefore the best timing of treatment for a patient may be missed and the ineffective treatment and dosing scheme cannot be adjusted timely and effectively. As a result, not all the metastasis tumors can be treated successfully, and finally the death of the patient occurs. In a clinical perspective, the metastasis tumor can be seen as the conclusive event for a natural progression of cancer.

People are now in urgent need of development of a tool for non-invasively sampling the tumor cells from a patient.

Circulating tumor cells (CTCs) refer to live solid tumor cells existing at a rather low level in the blood. With the development of researching on the circulating tumor cells, enrichment and identification of this kind of cells have become an adjuvant process for cancer diagnosis. Some regulatory agencies (such as FDA) have approved a number of clinical applications based on the capture and identification system of circulating tumor cells.

In order to separate and enrich the circulating tumor cells and disseminated tumor cells in body fluid, some capture and enrichment methods have been developed based on the characteristics of these cells such as the cell size, and the expression of surface molecules (e.g. porous filter membrane method, immunomagnetic enrichment method and the like).

The traditional porous filter membrane method has the following disadvantages: (1) the filter membrane has a single pore size which does not conform to various cell sizes of clinical patients, as a result, some cells may be missed; (2) this method has large reagent consumption, it is difficult to carry out the subsequent identification process; (3) the filtration pores are easy to block, which has an effect on the experimental result; (4) the preparation process of the special membrane is complex and has high cost.

The immune recognition and enrichment process mainly includes the following two methods: enrichment by magnetic beads and enrichment by chip. Because of the complexity and diversity of organisms, the trait recognition group of tumor cells in body fluid may have the possibility to degrade, as a result the immune recognition efficiency decreases, and false negative or false positive occurs. Both enrichment by magnetic beads and enrichment by chip have the disadvantages of low probability of the contact between the cell and immune recognition group, and weak combination, which impact the final detection and diagnosis. Meanwhile, this kind of chip has high cost, and is difficult to manufacture. Accordingly, it is necessary to make improvement on the current separation and enrichment method of circulating tumor cells.

### SUMMARY

The technical problem to be solved by the present application is to provide a microfluidic cell capture chip for separation, and enrichment of rare cells from human body fluid, so as to solve the problems with invasive sampling in prior art.

The technical solution for solving the technical problem of the present application is: providing a microfluidic cell capture chip comprising an upper rigid material, a lower rigid material and a channel formed between the upper and lower rigid materials, wherein the channel has an inlet and an outlet, at least one of the upper and lower rigid materials is a transparent material, the channel has a height gradually decreasing from the inlet to outlet and has a shape of wedge or a portion of the channel has a shape of wedge, and the lowest part of the channel has a dimension approximate to or less than the size of at least one of the target cells.

As an improvement of the present application, the channel has a width of 0.05-200 millimeters and a length of 1-500 millimeters.

As a further improvement of the present application, each of the upper and lower bottom surface of the channel has a nanoparticle layer or a nanofiber layer or a micro/nano structure deposited thereon, wherein the micro/nano structure is used for increasing the friction between the cells and the contact surface, and the nanoparticle layer or nanofiber layer is made of nano TiO₂, SiO₂ or Fe₂O₃.

As a further improvement of the present application, at least one surface of the said upper and lower rigid materials is immune modified so as to molecular specifically recognize at least one target cell.

As a further improvement of the present application, there is a steel sheet with the thickness of 50-200 microns squeezed at the inlet of the channel between the upper and lower rigid materials, and a steel sheet with the thickness of 1-50 microns is arranged at the outlet of the channel between the upper and lower rigid materials, and the channel is formed between the two steel sheets.

As a further improvement of the present application, both the upper rigid material and the lower rigid material are made of glass or acrylic material.

Another technical solution for solving the technical problem of the present application is: providing a method for producing a microfluidic cell capture chip comprising:

Step One: overlapping an upper rigid material on a lower rigid material;

Step Two: squeezing a thick steel sheet into one overlapped end between the upper and lower rigid materials and clamping the upper and lower rigid materials at this end with a clamping tool, then squeezing a thin steel sheet into the other overlapped end between the upper and lower rigid materials and clamping the upper and lower rigid materials at this end with a clamping tool, so as to form a channel with a wedge shape between the upper and lower rigid materials;

Step Three: sealing the side slits between the upper and lower rigid materials with polydimethylsiloxane, then drying, so that the body fluid sample cannot flow out of the sides of the upper and lower rigid materials;

Step Four: sealing both ends of the wedge shaped channel with polydimethylsiloxane, then drying, arranging a through hole at the position of the thick steel sheet to form an inlet of the wedge shaped channel, arranging another through hole at the position of the thin steel sheet to form an outlet of the wedge shaped channel, so that a body fluid sample can flow into the wedge shaped channel from the inlet and out of the channel from the outlet.

As an improvement of the method according to the present application, the method further comprises: Step Five: inserting a vent needle into each of the inlet and the outlet of the wedge shaped structure so as to let the body fluid sample pass through the vent needle.

As a further improvement of the method according to the present application, the thick steel sheet has a thickness of 50-200 microns, and the thin steel sheet has a thickness of 1-50 microns.

As a further improvement of the method according to the present application, the channel has a width of 0.05-200 millimeters and a length of 1-500 millimeters.

As a further improvement of the method according to the present application, in Step One, each of the upper and lower bottom surfaces has a nanoparticle layer or a nanofiber layer or a micro/nano structure deposited thereon, wherein the micro/nano structure is used for increasing the friction between the cells and the contact surface, and the nanoparticle layer or nanofiber layer is made of nano TiO₂, SiO₂ or Fe₂O₃.

As a further improvement of the method according to the present application, at least one surface of the upper and lower rigid materials is immune modified so as to molecular specifically recognize at least one target cell.

As a further improvement of the method according to the present application, the process for modifying the at least one surface is: Step One: preparing 4% solution of 3-mercaptopropyl trimethoxysilane in absolute ethanol, filling the channel of a chip with this solution, after reaction for 1 hour at room temperature rinsing this channel with absolute ethanol for 5 minutes; Step Two: preparing 1 μmol/mL solution of a protein crosslinker 4-maleimidobutyric acid-N-succinimide ester in dimethylsulfoxide, injecting the obtained solution into the channel of the chip, after reaction for 45 minutes at room temperature rinsing the channel with absolute ethanol for 5 minutes; Step Three: preparing 50 μg/mL solution of streptavidin-biotin in phosphate buffer solution, injecting the obtained solution into the channel of the chip, placing this chip in 4° C. refrigerator overnight for reaction, then washing the channel with phosphate buffer solution with pH=7.2-7.4 for 5 minutes; Step Four: injecting a solution of epithelial cell adhesion molecule antibody into the channel of the chip, standing at room temperature for 1-2 hours then washing the channel with PBS for 5 minutes.

As a further improvement of the method according to the present application, both the upper rigid material and the lower rigid material are made of glass or acrylic material.

According to the present application, a body fluid sample can be obtained from a patient in non-invasive way, and the body fluid sample is injected into the chip from the inlet of the channel. Because the height of the micro channel has a shape of wedge, the target cells may be separated and enriched automatically as they are passing through the channel. The microfluidic cell capture chip according to the present application has a simple structure, it is easy to produce and has low cost, the chip can separate and enrich cells with different sizes and specific molecular expression rapidly and efficiently.

### BRIEF DESCRIPTION OF THE DRAWINGS

The present application will be described in detail in the following description with reference to the accompanying drawings and examples of which:

FIG. 1 is a structural schematic diagram of a microfluidic cell capture chip according to an embodiment of the present application;

FIG. 2 is an enlarged diagram in a longitudinal section of a microfluidic cell capture chip according to an embodiment of the present application;

FIG. 3 is a schematic diagram showing the separation of cells with the microfluidic cell capture chip according to the present application.

### DETAILED DESCRIPTION OF THE INVENTION

Objects advantages and embodiments of the present application will be explained below in detail with reference to the accompanying drawings and examples. However it should be appreciated that the following description of the example is merely exemplary in nature and is not intended to limit this application.

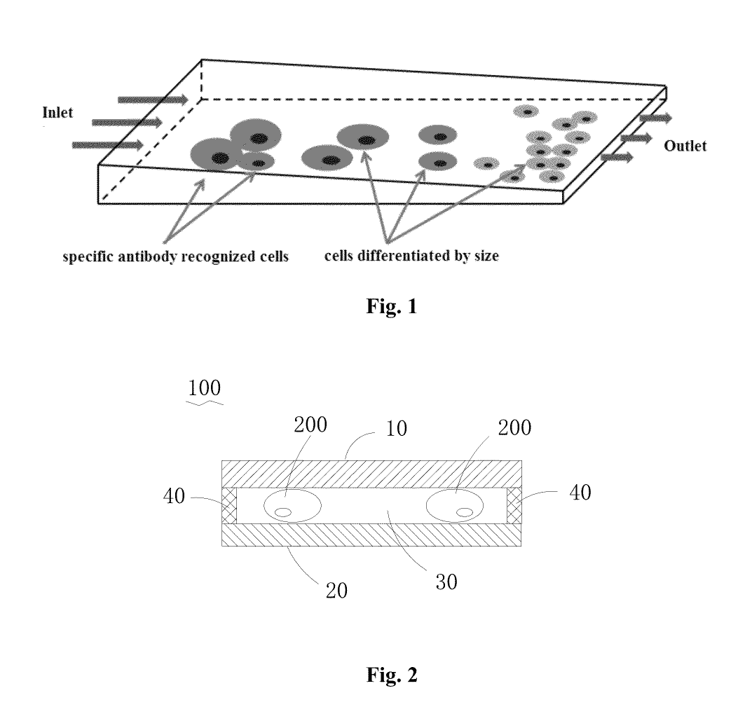

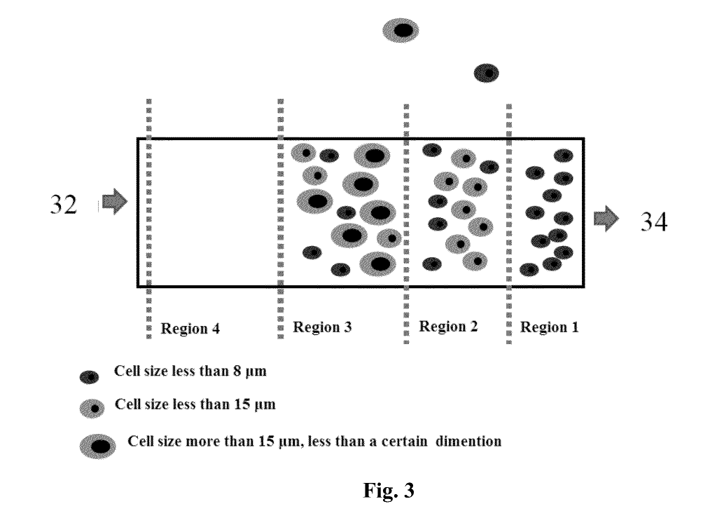

As shown in FIGS. 1-3, the embodiments of the present application provide a microfluidic cell capture chip 100 for capturing microfluidic cells 200. This microfluidic cell capture chip 100 includes an upper transparent glass sheet 10, a lower transparent glass sheet 20 and a channel 30 formed between the upper and lower transparent glass sheets 10, 20, wherein the channel 30 has an inlet 32 and an outlet 34, the channel 30 has a height decreasing gradually from the inlet 32 to the outlet 34 and the channel has a shape of wedge or a portion of the channel has a shape of wedge, and the lowest part of the channel 30 is approximate to or less than the size of at least one target cell. In this embodiment, the microfluidic cell 200 is circulating tumor cell. In this embodiment, it is not limited to the transparent glass sheet, and these transparent glass sheets can be replaced with other transparent rigid materials, such as acrylic material. Furthermore, the upper and lower transparent glass sheets can be replaced with a transparent rigid material and a nontransparent rigid material, that is to say, it is also suitable that only one of the two sheets is a transparent rigid material.

The channel 30 has a width of 0.05-200 millimeters and a length of 1-500 millimeters. At least one surface of the upper transparent glass sheet 10 and lower transparent glass sheet 20 can be surface modified. The process for surface modifying at least one surface is: Step One: preparing 4% solution of 3-mercaptopropyl trimethoxysilane (MPTMS) in absolute ethanol and filling this solution into the channel of a chip, after reaction at room temperature for 1 hour, rinsing this channel with absolute ethanol for 5 minutes; Step Two: preparing 1 μmol/mL solution of a protein crosslinker 4-maleimidobutyric acid-N-succinimide ester (GMBS) in dimethylsulfoxide (DMSO), injecting the obtained solution into the channel of the chip, after reaction for 45 minutes at room temperature, rinsing the channel with absolute ethanol for 5 minutes; Step Three: preparing 50 μg/mL solution of streptavidin-biotin (SA) in phosphate buffer solution (PBS) and injecting the obtained solution into the channel of the chip, after placing this chip in 4° C. refrigerator overnight for reaction, washing the channel with PBS (phosphate buffer solution, pH=7.2 to 7.4) for 5 minutes; Step Four: injecting a solution of epithelial cell adhesion molecule antibody (Anti-EpCAM) into the channel of the chip, standing at room temperature for 1-2 hours then washing the channel with PBS for 5 minutes. After the surface modification, it includes specific antibody that molecularly recognizes at least one target cell. There is a steel sheet 40 with the thickness of 50 to 200 microns squeezed in the inlet 32 of the channel 30 between the upper transparent glass sheet 10 and lower transparent glass sheet 20, and a steel sheet 40 with the thickness of 1-50 microns is squeezed in the outlet 34 of the channel 30 between the upper transparent glass sheet 10 and lower transparent glass sheet 20, and the channel is formed between the two steel sheets 40.

According to the embodiments of the present application, a microfluidic cell capture chip for the separation and enrichment of cells from human body fluid sample is provided, wherein the channel 30 in the microfluidic cell capture chip 100 has a regularly changed height, and the upper and lower bottom surfaces of the channel 30 is specially processed, i.e. a layer of TiO₂, SiO₂ or Fe₂O₃ as a nano-film or nanofiber or micro/nano structure facilitating to increase the friction between the cells and the contact surface is deposited on the upper and lower surfaces of the channel, and the layer has a thickness of 5-200 nanometers, so as to improve the efficiency of target cell capture. The microfluidic cell capture chip according to the present application has a simple structure, it is easy to produce and has a low cost, and this chip can separate and enrich cells with different sizes and specific molecular expression rapidly and efficiently.

The microfluidic cell capture chip 100 according to the present application can separate and enrich the cells in non-manual way. The cells are separated and enriched automatically as the fluid flows through the microfluidic cell capture chip. For the microfluidic cell capture chip according to the present application, the cells are marked with at least one kind of tracers so they can be recognized by this chip. According to the present application, a body fluid sample is obtained from a patient in non-invasive way, and the body fluid sample is injected into the microfluidic cell capture chip 100 from the inlet 32. Because the height of the channel 30 distributes in a shape of wedge, the target cells may be separated and enriched automatically as they are passing through the channel 30.

The present application further provides a manufacture method of a microfluidic cell capture chip comprising:

Step One: overlapping an upper transparent glass sheet 10 on a lower transparent glass sheet 20;

Step Two: squeezing a precision steel sheet 40 of 50-200 micron thickness into one end of the two overlapped glass sheets, and clamping the glass sheets at the overlapped end with a clamping tool, squeezing another precision steel sheet of 1-50 micron thickness into the other end the overlapped glass sheets and clamping the glass sheets at this end with a clamp tool, so as to form a wedge shaped channel 30 between the two glass sheets;

Step Three: sealing both side slits of the two glass sheets with a polydimethylsiloxane (PDMS), then drying on a warm table, accordingly the body fluid sample cannot flow out from any side of the glass sheet;

Step Four: sealing both ends of the wedge shaped channel with polydimethylsiloxane and drying, punching a hole on each end of the channel so that the body fluid sample can flow into the wedge shaped channel 30 from the inlet 32 and out of the channel 30 from the outlet 34, as shown in FIG. 1.

Step Five: inserting a vent needle into each of the inlet 32 and outlet 34 of the wedge shaped channel 30 as shown in FIG. 1 for the flow through of the body fluid sample, so that the microfluidic cell capture chip is completed.

Experimental Principle:

Referring to FIG. 1, the present application employs a wedge shaped channel structure, and the channel formed between the two glass sheets has a width of 0.05-200 millimeters, and a length of 10-500 millimeters.

The wedge shaped channel in the chip according to the present application has an inlet height of 50-200 microns and an outlet height of 1 −50 microns, when a body fluid sample entries the inlet and flows through the sedge shaped channel, the target cells in the body fluid sample will be captured at a special position of the channel because of the restriction of the fluid space.

The basic idea of this experiment is passing the body fluid sample from a patient to be detected through the wedge shaped microfluidic capture chip according to the present application, and finally separating and enriching the target cells with various sizes automatically when they are passing through the channel.

Experimental Procedure:

(1) A microfluidic circulating tumor cell capture chip of wedge shape as shown in FIG. 1 is made according to the manufacture method of the present application;

(2) injecting a body fluid sample to be tested into the chip from the inlet, collecting the body fluid sample after separation of target cells at the outlet of the chip;

(3) adding phosphate buffer solution (i.e. PBS solution, PH=7.2-7.4, NaCl 137 mmol/L, KCl 2.7 mmol/L, Na₂HPO₄ 4.3 mmol/L, KH₂PO₄ 1.4 mmol/L) from the inlet for rinse;

(4) selectively adding tracers (such as: fluorescent dyes DAPI or Hoechst dye for nucleus staining) or immunological reagents from the inlet to identify the target cells;

(5) adding PBS from the inlet again for rinse;

(6) placing the chip under a microscope to observe the captured target cells.

Analysis of the Experimental Results

(1) Referring to FIG. 2, four observation points in region 1, region 2, region 3 and region 4 respectively are selected, and the results of separation and enrichment of the target cells by the chip can be observed.

(2) It can be seen from the distribution of target cells at the four observation points of the four regions that, the separation and enrichment of the target cells are the result of joint action of target cell sizes and the wedge shaped channel size, wherein the cells of larger size are enriched away from the outlet, while the cells of smaller size are enriched adjacent to the outlet.

(3) This experiment can achieve the separation and capture of the target cells with various sizes, and it has a simple and repeatable process.

(4) The microfluidic cell capture chip of wedge shape employed in this experiment has a simple structure, it is easy to produce and has a low cost, this chip can separate and enrich the cells with various sizes and specific molecular expression rapidly and efficiently.

According to the present application, a body fluid sample can be obtained from a patient in non-invasive way and the body fluid sample is injected into the micro channel chip from the inlet. Because the height of the micro channel distributes in a shape of wedge the target cells can be separated and enriched automatically when they are passing through the channel. The microfluidic cell capture chip according to the present application has a simple structure, it is easy to produce and has low cost, it is suitable for separating and enriching cells with different sizes and specific molecular expression rapidly and efficiently.

The embodiments above are merely the preferable embodiments of the present application and not intended to limit the present application. And all changes equivalent substitution and improvements which come within the meaning and range of equivalency of the present application are intended to be embraced therein.

1. A microfluidic cell capture chip for separation, enrichment and recognition of circulating tumor cells comprising: an upper rigid material, a lower rigid material, and a channel formed therebetween, wherein the channel has an inlet and an outlet, at least one of the upper and lower rigid materials is a transparent material,
characterized in that, the channel has a height gradually decreasing from the inlet to outlet and has a shape of wedge, or a portion of the channel has a shape of wedge, and the lowest part of the channel has a dimension approximate to or less than a size of a circulating tumor cell,
each of upper and lower bottom surfaces of the channel has a nanoparticle layer or a nanofiber layer deposited thereon, the nanoparticle layer or nanofiber layer is made of nano TiO₂, SiO₂ or Fe₂O₃, and at least one surface of the upper and lower rigid materials is immune modified so as to molecular specifically recognize the circulating tumor cell.

2. The microfluidic cell capture chip of claim 1, wherein the channel has a width of 0.05-200 millimeters and a length of 1-500 millimeters.

3. (canceled)

4. (canceled)

5. (canceled)

6. The microfluidic cell capture chip of claim 1, wherein there is a first steel sheet with a thickness of 50 to 200 microns arranged at the inlet of the channel between the upper and lower rigid materials and a second steel sheet with the thickness of 1 to 50 microns arranged at the outlet of the channel between the upper and lower rigid materials, and the channel is formed between the two steel sheets.

7. The microfluidic cell capture chip of claim 1, wherein both the upper rigid material and the lower rigid material are made of glass or acrylic material.

8. A method for manufacturing a microfluidic cell capture chip for separation, enrichment and recognition of circulating tumor cells, wherein the method comprises:
Step One: overlapping an upper rigid material on a lower rigid material, wherein each surface of the upper and lower rigid materials has a nanoparticle layer or a nanofiber layer, the nanoparticle layer or nanofiber layer is made of nano TiO₂, SiO₂ or Fe₂O₃;
Step Two: squeezing a thick steel sheet into one end between the upper and lower rigid materials and clamping the overlapped upper and lower rigid materials at the end with a clamping tool, squeezing a thin steel sheet into another end between the upper and lower rigid materials and clamping the overlapped upper and lower rigid materials at the another end with a clamping tool, so as to form a channel with wedge shape between the upper and lower rigid materials, and the lowest part of the channel has a dimension approximate to or less than a size of a circulating tumor cell;
Step Three: sealing the side slits between the upper and lower rigid materials with polydimethylsiloxane, drying, so that a body fluid sample cannot flow out of the sides of the upper and lower rigid materials;
Step Four: sealing both ends of the wedge shaped channel with polydimethylsiloxane, drying, arranging a through hole at the position of the thick steel sheet to form an inlet of the wedge shaped channel, arranging another through hole at the position of the thin steel sheet to form an outlet of the wedge shaped channel, so a body fluid sample can flow into the wedge shaped channel from the inlet and out of the channel from the outlet, at least one surface of the upper and lower rigid materials is immune modified in the method so as to molecular specifically recognize the circulating tumor cell.

9. The method of claim 8, wherein the method further comprises: Step Five: inserting a vent needle into each of the inlet and the outlet of the wedge shaped structure to let a body fluid sample pass through.

10. The method of claim 8, wherein the thick steel sheet has a thickness of 50 to 200 microns, and the thin steel sheet has a thickness of 1 to 50 microns.

11. The method of claim 8, wherein the channel has a width of 0.05 to 200 millimeters and a length of 1 to 500 millimeters.

12. (canceled)

13. (canceled)

14. (canceled)

15. The method of claim 8, wherein the process for surface modifying the at least one surface is:
Step One: preparing 4% solution of 3-mercaptopropyl trimethoxysilane in absolute ethanol, filling the channel of a chip with the obtained solution, after reaction for 1 hour at room temperature rinsing this channel with absolute ethanol for 5 minutes;
Step Two: preparing 1 μmol/mL solution of a protein crosslinker 4-maleimidobutyric acid-N-succinimide ester in dimethylsulfoxide, injecting the obtained solution into the channel of the chip, after reaction for 45 minutes at room temperature rinsing the channel with absolute ethanol for 5 minutes;
Step Three: preparing 50 μg/mL solution of streptavidin-biotin in phosphate buffer solution, injecting the obtained solution into the channel of the chip, placing the chip in 4° C. refrigerator overnight for reaction, then washing the channel with phosphate buffer solution with pH 7.2 to 7.4 for 5 minutes;
Step Four: injecting a solution of epithelial cell adhesion molecule antibody into the channel of the chip, standing at room temperature for 1 to 2 hours, then washing the channel with PBS for 5 minutes.

16. The method of claim 8, wherein both the upper rigid material and the lower rigid material are made of glass or acrylic material.

17. A microfluidic cell capture chip made by the method of claim 8.
